(19) United States
(12) Patent Application Publication
(10) Pub. No.: US 2026/0298927 A1
(43) Pub. Date: Oct. 1, 2026

(54) BIOSENSORS AND METHODS FOR THE DETERMINATION OF THE MONKEYPOX AND CHICKENPOX VIRUSES BELONGING TO THE GENUS ORTHOPOXVIRUS

(71) Applicant: MUGLA SITKI KOCMAN UNIVERSITESI STRATEJI GELISTIRME DAIRE BASKANLIGI, Mugla (TR)

(72) Inventors: Ulku ANIK, Mugla (TR); Yudum TEPELI BUYUKSUNETCI, Mugla (TR)

(73) Assignee: MUGLA SITKI KOCMAN UNIVERSITESI STRATEJI GELISTIRME DAIRE BASKANLIGI, Mugla (TR)

(21) Appl. No.: 19/475,757

(22) PCT Filed: Mar. 1, 2024
(86) PCT No.: PCT/TR2024/050178

§ 371 Date: Oct. 16, 2025

(30) Foreign Application Priority Data
Apr. 17, 2023 (TR) 2023/004290

(51) Int. Cl. G01N 33/569; G01N 21/78; G01N 33/543

(52) U.S. Cl. CPC G01N 33/56983; G01N 21/78; G01N 33/5438; G01N 2333/04; G01N 2333/065

(57) ABSTRACT

A biosensor is provided, which has been developed for the diseases caused by monkeypox and chickenpox viruses with reference to their infection mechanisms and may be used for the diagnosis thereof, and a method for the determination of possible monkeypox and chickenpox viruses in individuals using the operating system of these biosensors is also provided.

### CROSS REFERENCE TO THE RELATED APPLICATIONS

This application is the national phase entry of International Application No. PCT/TR2024/050178, filed on Mar. 1, 2024, which is based upon and claims priority to Turkish Patent Application No. 2023/004290, filed on Apr. 17, 2023, the entire contents of which are incorporated herein by reference.

### TECHNICAL FIELD

The invention relates to biosensors which have been developed with reference to the infection mechanisms of diseases caused by monkeypox and chickenpox (also known in the art as variola) viruses belonging to the genus Orthopoxvirus and are suitable for use in the diagnosis thereof.

The invention based on the biosensors that were developed with reference to the infection mechanisms of diseases caused by monkeypox and chickenpox viruses belonging to the genus Orthopoxvirus. It's aimed to provide self testing or to be used in in a health center where a patient can get the test results in a short time.

The diagnostic method of the invention is carried out by in-vitro methods.

### BACKGROUND

Monkeypox is a disease caused by the Monkeypox virus, which belongs to the virus family Poxviridae and the genus Orthopoxvirus. Although the name of the disease is monkeypox, the virus is actually of rodent origin. The virus, which was detected in 1958 as a result of two different outbreaks with symptoms similar to smallpox in monkey colonies kept for research, was therefore called monkeypox. In humans, an infection with the monkeypox virus was first observed in 1970. From those years to the present day, monkeypox has been seen regularly as rare cases on the African continent. Monkeypox, which has been seen for many years in West and Central Africa, where tropical rainforests are abundant, and has been limited to this region, rarely reached different parts of the world, with transmission caused by animals exported from the region. However, in the recent period, the disease has become more widespread than in the past and has been diagnosed in different people from different regions. Monkeypox cases, which are seen as a possible new virus outbreak due to the devastating effects of the Covid-19 pandemic on the world, have started to be followed in detail, even though they has not showed an epidemic spread.

Monkeypox may be transmitted to humans through an animal infected with the virus, or through another person who has had a monkeypox infection. As with any virus, monkeypox has an incubation period after contact with the infectious agent. The first symptoms may manifest any time from 5 to 21 days. The most common onset of the symptoms was found to be between 6-13 days. Although the symptoms are similar to smallpox, which has caused large epidemics in the past, it progresses slightly differently. The symptoms of monkeypox virus disease include high fever, headaches, backaches and muscle aches, swollen lymph nodes, exhaustion, chills and rigors, and rashes on the skin in the form of small blisters filled with water similar to chickenpox.

The laboratory-validated diagnostic methods for monkeypox, which is currently very likely to turn into a pandemic in the world and the early detection of which has become very important, include electron microscopy, isolation of the virus on chorioallantonic membranes (also abbreviated as CAM), antibody-based assays and viral DNA assays. Of these, only CAM and DNA assays may provide a species-specific diagnosis. These diagnostic techniques require sensitive reagents and equipment.

On the other hand, chickenpox is an acute infectious disease caused by the variola virus, which is also a species of the genus Orthopoxvirus. Chickenpox disease believed to have been around for at least 3000 years, is one of the most devastating diseases known to Mankind and caused millions of deaths before it was eradicated.

The smallpox vaccine, developed by Edward Jenner in 1796, was the first successful vaccine developed. Thanks to this, chickenpox was eradicated, and the last known natural case was in Somalia in 1977. However, the fact that the smallpox vaccine is not applied as widely as in the past increases the possibility of chickenpox recurring, as well as monkeypox.

An antibody-based serological assay for the diagnosis of monkeypox and chickenpox is ideal for the assay, diagnosis and surveillance, but due to the antigen protection within Orthopoxvirus genomes, most sera are cross-reactive and cannot distinguish between the individual Orthopoxvirus species. Of the other techniques applied for the diagnosis of monkeypox and chickenpox diseases, polymerase chain reaction (PCR) is the laboratory test of choice considering its accuracy and sensitivity. Therefore, the most suitable diagnostic samples for monkeypox and chickenpox are obtained from skin lesions, the roof or fluid from vesicles and pustules and dry crusts. Some of the PCR, CAM and DNA analyses are not highly accurate, and all these tests are time-consuming techniques. In addition, such techniques should be performed by the skilled ones in the art and involve expensive instrumentation. Especially for the monkeypox virus, which has a potential to turn into a pandemic, much more practical, fast and economical tests are needed.

Consequently, there is a need for researching and developing safe, fast and economical test methods which do not require expertise in possible pandemic conditions for monkeypox and chickenpox diseases in order to provide solutions and advantages to the needs of the relevant technical field.

### SUMMARY

The pandemic created by the monkeypox virus around the world has caused great destruction for people and has created drawbacks in many ways. On the other hand, chickenpox has caused millions of losses over the years. For monkeypox patients, early diagnosis and treatment of the disease and quarantine are very important under the pandemic conditions. For example, the coronavirus disease, which has emerged recently and cannot be diagnosed early, has spread rapidly and caused a pandemic. Current testing methods mean that monkeypox cannot be addressed in a quick, safe and economical manner. The same applies to chickenpox, since the widespread administration of smallpox vaccine has ended.

Similarly, it is aimed to avoid technical problems, especially for monkeypox, which has a pandemic potential. The present inventors provide biosensors which is reliable, fast, economical for the elimination of the technical problems related to the monkeypox virus during pandemics and the detection of the monkeypox and chickenpox viruses and which also allows diagnosis without a skilled one in the art, and a method for the use of said biosensors in the diagnosis of monkeypox and chickenpox diseases.

### DETAILED DESCRIPTION OF THE EMBODIMENTS

In this detailed explanation, the subject of the invention is related to biosensors which enable the diagnosis of diseases caused by monkeypox and chickenpox (variola) viruses belonging to the genus Orthopoxvirus under in-vitro conditions and the methods of said biosensors for the diagnosis of diseases caused by monkeypox and chickenpox (variola) viruses, and is described with examples only for a better understanding of the subject in a non-limiting sense. Said methods and biosensors allow to diagnose the possible presence of monkeypox and chickenpox viruses in samples. The biosensor and method used include very simple equipment, and the patient may perform the tests in short times by himself/herself, or in a health center and analysis the results. The method used in the invention is carried out under in-vitro conditions.

The biosensors of the invention generally contain two main components. One of said components is at least one bioreceptor which may react with the possible monkeypox or variola viruses in the sample by a biochemical reaction, and another component is at least one sensor which converts a biological event taking place in at least one bioreceptor into significant expressions.

In this invention, the presence of viruses which are the source of monkeypox and chickenpox diseases is diagnosed from the samples. This is mainly provided by at least one bioreceptor in the biosensor. Said bioreceptor significantly reacts with the virus which is the source of monkeypox and/or chickenpox in the sample, thereby forming at least one biomolecular structure. Bioreceptors, the most important components for the biosensor of the invention, may be defined as a biological molecule which may selectively interact with the virus that is the source of monkeypox and chickenpox in the sample.

The biosensor of the invention contains at least one bioreceptor, while said bioreceptor may be at least one of the proteins, enzymes, antibodies, nucleic acids, cells, tissues and/or microorganisms.

The basic principle in the invention is that the individuals may diagnose the viruses that are the source of monkeypox and chickenpox by means of the interaction of the biological active substance to be used as a bioreceptor with the virus that is the source of monkeypox and chickenpox, and the sensors that convert this interaction into significant expressions upon a biological event.

In this invention, the biosensor contains at least one glucosaminglycan (abbreviated as GAG) polysaccharide as a bioreceptor. GAGs are known as long, polysaccharide molecules (glycans) that are inherent in the body. A GAG compound is called a compound consisting of different disaccharide molecules. These compounds include sugar units such as N-acetylglucosamine and glucoronic acid. In this invention, heparan sulfate polysaccharide preferably may be used as GAG. If preferred, it contains, as GAG, at least one of the polysaccharides heparin, chondroitin-4-sulfate, chondroitin-4,6-sulfate, and dermatan sulfate, which have a higher degree of sulfation than heparan sulfate polysaccharide.

The interaction of the bioreceptor used in the invention with monkeypox viruses has been proved in the studies. This interaction is based on the proteins A29 and A27 in monkeypox. In particular, the A29 protein is recognized as the specific protein which allows for the specific diagnosis of monkeypox. In this context, one of the above-mentioned GAGs to be attached to the biosensor reacts with the A29 protein on the surface of the monkeypox virus, thereby providing the biological interaction necessary for the biosensor to work. In addition, the A27 protein on the surface of the variola virus reacts with one of the GAGs mentioned above and similarly provides the basic interaction in terms of the biosensor.

Said biosensor contains at least one sensor which converts the biological expressions resulting from the bioreceptor-virus interaction into significant signals.

The biosensor of the invention may have two working methods according to the working principle of the sensor. In the invention, the electrochemical diagnosis method of monkeypox and chickenpox viruses,
in which the biological event obtained from bioreceptor-monkeypox and bioreceptor-chicken pox virus is converted into electrochemical expressions as a significant expression,
or the colorimetric diagnosis method of monkeypox and chickenpox viruses, in which a color change is taken into account as a significant expression, may be used.

In the future, a hybrid method in which these two methods are combined may be included in the scope of protection of the invention.

Accordingly, if the biosensor of the invention
diagnoses monkeypox and chickenpox viruses by the electrochemical methods, it contains
   at least one sample taken from the suspicious person, from which the tests will be carried out,
   at least one bioreceptor which may interact with possible monkeypox and chickenpox viruses in the sample,
   at least one transducer which converts the biological interaction occurring when it interacts with possible monkeypox and chickenpox in the bioreceptor-sample, into significant electrical expressions;
if it diagnoses monkeypox and chickenpox viruses by the colorimetric methods, it contains
   at least one sample taken from the suspicious person, from which the tests will be carried out,
   at least one bioreceptor which may interact with possible monkeypox and chickenpox viruses in the sample,
   at least one color-changing reactive compound which converts the biological event occurring when it interacts with possible monkeypox and chickenpox in the bioreceptor-sample into the significant color changes.

In the invention, the sample required to diagnose the presence of monkeypox and chickenpox viruses in the biosensor is preferably a sample taken from a wound in the human body, or serum samples if suspected before the wounds appear, which will allow early diagnosis.

As mentioned before, the biosensor of the invention may be applied using colorimetric or electrochemical methods, depending on the interpretation of the resulting biological expressions.

The colorimetric method may be chosen as the working method of the biosensor of the invention. In the colorimetric method, it is possible to interpret the biological event obtained in the biosensor with a color change. Therefore, there is at least one color-changing reagent in the biosensor, which will provide said color change. The color change may be detected by a spectrophotometer, or only a human eye. At least one color-changing reactive compound selected from the group consisting of a gold nanoparticle, 2,2 azino-bis (3-ethyl benzothiazolin- 6-sulfonic acid (abbreviated as ABTS) and 3,3′5,5′-tetramethylbenzidine (abbreviated as TMB) is used as the color changing reactive compound to obtain the color change.

If at least one of the ABTS and TMB compounds is used, additional reagents (both H₂O₂ and an additional oxidizing reagent) should be added to the medium to ensure color formation. In this invention, the oxidizing color-changing reactive compound includes reagents that have oxidizing characteristics against the reagents such as “TMB or ABTS”. Examples include oxido-reductase enzymes as oxidizing reagents, or metal-organic skeleton (MOF) or nanomaterials (such as Fe2O3 nanoparticles, etc.) which mimic that characteristic. However, the scope of protection of the invention is not limited to the use of oxido-reductase enzymes and reagents mimicking them as the oxidizing reagents.

This is based on determination of the presence of monkeypox or chickenpox virus by obtaining a color different from the current color of the color-changing reactive compound that will interact with the biomolecule obtained from the bioreceptor-monkeypox and/or bioreceptor-chickenpox virus interaction. Accordingly, there should be at least one color-changing reagent in said biosensor. When a gold nanoparticle nanomaterial is used as the color-changing reactive compound used in this invention, the gold nanoparticle, which is initially red, turns into different shades of red, or purple, upon the bioreceptor-monkeypox or chickenpox virus interaction. If, on the other hand, at the beginning, TMB or ABTS together with additional reagents, instead of the gold nanoparticle, blue and green colors are observed, respectively. Upon interaction, these colors turn into light blue and light green, and sometimes this transformation continues until a colorless solution is obtained.

The electrochemical method may be selected as the working method of the biosensor of the invention. In the electrochemical method, the biological event obtained in the biosensor is interpreted with the electrical expressions. Therefore, there is provided at least one transducer in the biosensor, which allows the conversion of the obtained biological interaction into the electrical expressions. This is based on the conversion of the biological event formed by the biomolecule obtained from the bioreceptor-monkeypox or bioreceptor-chickenpox virus interaction into the electrical expressions. In this embodiment, there is provided an electrode as a transducer which may perform the electrochemical measurement. The bioreceptors to be used in the invention are immobilized on commercially available electrodes by the crosslinkers such as 1-ethyl-3-(3-dimethylaminopropyl)carbodiimide hydrochloride/N-hydroxysuccinimide (abbreviated as EDC/NHS). In the sample taken from the patient, an appropriate bioreceptor will interact with the source protein (A29 or A27) of monkeypox or chickenpox. As a result, the impedance and resistance of the electrode will increase, or the current obtained from that electrode will decrease. While this impedance and increase in resistance are examined by the electrochemical impedance spectroscopy (EIS) method, the decrease in current may be monitored by various voltammetric techniques (cyclic voltammetry (CV), differential pulse voltammetry (DPV), squarewave voltammetry (SWV)). The structure in which the electrochemical method will be used as a transducer in the biosensor is obtained as follows: Generally, the screen printed, gold-or carbon-based electrodes may be used as the electrodes mentioned here. Apart from these, similar paper-based electrodes may also be preferred for this purpose. Various working conditions are optimized by monitoring the impedance change on the electrode surface by EIS, or by monitoring the current decay by the voltammetric techniques mentioned above. Then, analytical characteristics are examined.

Whether the biosensor of the invention performs measurements by a colorimetric method, or electrochemical transducers, the basic reaction is provided by the reaction of the bioreceptors with proteins that are the source of monkeypox or chickenpox. The biological event here is based on the binding of the A27 or A29 proteins on viruses to at least one of the bioreceptors. The presence of such an interaction is certain, and by measuring the biological event obtained, the patient will be able to take a swab from his/her own wound and understand whether he/she carries the source virus of monkeypox and chickenpox especially by means of the colorimetric system, without even requiring any specialist interpretation. Otherwise, in case of any suspicion in the first stage of the diseases, they will be able to admit to the nearest health unit and perform these tests on blood samples practically and quickly.

### Determination Method by the Biosensor

### Implementation of the Electrochemical Method

Blood samples or wound swabs of the individuals for whom the determination and measurement of monkeypox and chickenpox viruses are performed are taken on the electrode surface for the electrochemical biosensor. The changes occurred are monitored electrochemically.

Determination of monkeypox or chickenpox by an electrochemical biosensor includes the following process steps:
contacting the sample taken from the patient with the bioreceptors immobilized to the appropriate working electrode,
obtaining an interaction between the possible monkeypox or chickenpox viruses in the sample and the bioreceptors,
Diagnosing whether the individual is sick or not by monitoring the change in impedance, resistance or current as a result of the reactions occurring on the electrode surface.

The biosensor used contains at least one GAG polysaccharide as a bioreceptor. In this invention, heparan sulfate polysaccharide is preferably may be used as GAG. If preferred, it contains, as GAG, at least one of the polysaccharides heparin, chondroitin-4-sulfate, chondroitin-4,6-sulfate, and dermatan sulfate, which have a higher degree of sulfation than heparan sulfate polysaccharide. The bioreceptors are appropriately immobilized to the electrode surface. It uses crosslinkers such as EDC/NHS for the bioreceptor immobilization on the electrode surface. A29 protein on the monkeypox virus and A27 protein on chickenpox in thesample taken from the patient interact specifically with the bioreceptor. Upon this interaction, a protein layer originating from A29 or A27 is formed on the bioreceptor on the electrode surface. This layer makes the electron transfer on the electrode surface difficult. Thus, an increase in resistance or impedance value occurs in the electrochemical impedance measurements (known as EIS), or the decrease in current as a result of this protein layer is determined by various voltammetric techniques.

### Implementation of the Colorimetric Method

The swabs or blood samples taken from the wounds of the individuals for whom the measurements for the determination of monkeypox and chickenpox viruses are performed are taken into Eppendorf tubes containing color-changing reactive compounds. The changes occurred are examined qualitatively by the naked eye, and both qualitatively and quantitatively when a spectrophotometer is used.

The determination of monkeypox and chickenpox viruses by a biosensor using the colorimetry method is monitored by the following process steps:
firstly, adding the color-changing reactive compound (gold nanoparticle, TMB+H₂O₂+oxidizing reagent, or ABTS+H₂O₂+oxidizing reagent) to the Eppendorf tube, and then, adding the bioreceptor to the same Eppendorf tube in order to obtain red gold nanoparticle-bioreceptor solution if the gold nanoparticle is used, blue TMB-bioreceptor solution if TMB and additional reagents are used, and green ABTS-bioreceptor solution if ABTS and additional reagents are used,
darkening the red color and turning into different shades of red, or purple in relation with the analyte concentration upon addition of the sample obtained from the patient to the reagent solution and mixing, if a gold nanoparticle is used; gradually lightening the blue color, if TMB and additional reagents are used; and gradually lightening the green color, if ABTS and additional reagents are used.

When monkeypox or chickenpox viruses bind with the gold nanoparticle-labeled bioreceptors in the biosensor, a molecular growth and an increase in molecular weight occur in the groups attached to the gold nanoparticles. Therefore, the size of the gold nanoparticles stacked on top of each other increases. As different sizes of gold nanoparticles show different absorbance properties in the visible region, the color changes from red to different shades of red, or purple. If TMB or ABTS is used other than the gold nanoparticles, different oxidation and reduction reactions occur after the viruses are present in the medium, and as a result, the initial blue color in the presence of TMB is lightened, while the green color seen at the beginning in the presence of ABTS changes upon this interaction.

In this context, the color change indicates the presence of monkeypox or chickenpox virus in the sample taken from the patient.

The biosensor using the colorimetric method may be obtained by many configurations. The colorimetric biosensor may be considered not only in eppendorf tubes, but also on paper. Here, by adding a sample to the structure containing the bioreceptor and colored reagent solution on this paper, the patient will be able to easily realize whether he/she suffers from monkeypox or chickenpox diseases by a qualitative observation.

As can be seen, the biosensor obtained in the invention may be configured according to the reagents to be used. In this context, the interaction of the bioreceptor-monkeypox or chickenpox viruses and the subsequent measurement of the biological event obtained constitute the main basis of the invention. Therefore, any equipment or component (modified reagents, nanomaterials, etc.) that perfects the measurement of the aforementioned biological interaction may be added to the biosensor. Examples may include electronic signal amplifiers, data processing devices and additional auxiliary transducers. For example, if the electrochemical biosensor may be combined with a miniaturized potentiostat, a system suitable for the structure of the point-of-care (POC) may be obtained.

Thanks to the colorimetric biosensor obtained in the invention, the patient may easily test himself/herself. In this way, the patient intensity in a hospital caused by being tested may be reduced. In addition, the patient will be able to quarantine himself/herself without wasting time to reduce the rate of transmission. However, a blood testing may be done in the nearest health unit in a quick and practical manner at the early stage of the disease.

Thanks to the biosensor obtained in the invention, the cost, time and complexity of the tests will be reduced. In addition, the need for a specialist for testing is eliminated. As a result of reducing the cost, the test will be made more accessible. However, thanks to the biosensors adapted to the blood samples, the disease may be diagnosed at an early stage. Apart from these, the developed biosensors may be adapted to the determination of different viruses based on the infection steps by changing the necessary reagents.

What is claimed is:

1. A method for determining a presence of monkeypox virus and chickenpox virus in a sample, comprising qualitatively or quantitatively measuring a presence of possible monkeypox virus and possible chickenpox virus in the sample by electrochemical methods using a biosensor comprising at least one glycosaminoglycan (GAG), wherein the at least one GAG selectively interacts with the monkeypox virus and the chickenpox virus as a bioreceptor, and the method comprises the following process steps:
contacting the sample comprising the possible monkeypox virus or the possible chickenpox virus with the bioreceptor immobilized to an available working electrode,
obtaining a biological interaction between the possible monkeypox virus or the possible chickenpox virus in the sample and the bioreceptor, and
differentiating an impedance or a current on an electrode surface by the biological interaction obtained and ensuring an individual to understand whether the individual is sick or not by measuring a difference.

2. The method according to claim 1, wherein at least one of a gold-based surface printing electrode or a carbon-based surface printing electrode or paper electrode is used as the available working electrode.

3. The method according to claim 1, wherein a process of immobilizing the bioreceptor to a surface of the the available working_electrode is carried out with 1-ethyl-3-(3-dimethylaminopropyl)carbodiimide hydrochloride/N-hydroxysuccinimide (EDC/NHS), or different suitable crosslinking chemical compounds.

4. The method according to claim 1, wherein heparan sulfate is used as the at least one GAG.

5. The method according to claim 1, wherein the at least one GAG is at least one of polysaccharides of heparin, chondroitin-4-sulfate, chondroitin-4,6-sulfate, and dermatan sulfate.

6. A method for determining a presence of a monkeypox virus or a chickenpox virus, comprising qualitatively or quantitatively measuring a presence of a possible monkeypox virus or a possible chickenpox virus in a sample by colorimetric methods using a biosensor comprising at least one GAG, wherein the at least one GAG selectively interacts with the monkeypox virus and the chickenpox virus as a bioreceptor, and the method comprises the following process steps:
firstly adding a color-changing reactive compound to a reaction compartment,
adding an appropriate bioreceptor into the reaction compartment,
adding the sample obtained from a patient into the reaction compartment and stirring, and
detecting a presence or an absence of a color change in the reaction compartment.

7. The method according to claim 6, wherein the at least one GAG is heparan sulfate.

8. The method according to claim 6, wherein the at least one GAG is at least one of polysaccharides of heparin, chondroitin-4-sulfate, chondroitin-4,6-sulfate, and dermatan sulfate.

9. The method according to claim 6, wherein in a case of using gold nanoparticles as the color-changing reactive compound, a red-colored gold nanoparticle-bioreceptor solution turns into different shades of red, or purple.

10. The method according to claim 6, wherein in a case of using a 3,3′5,5′-tetramethylbenzidine (TMB)+H₂O₂+oxidizing reagent mixture as the color-changing reactive compound, a blue-colored TMB-bioreceptor solution turns from blue into a pale color.

11. The method according to claim 6, wherein in a case of using a 2,2 azino-bis (3-ethyl benzothiazolin- 6-sulfonic acid (ABTS)+H₂O₂+oxidizing reagent mixture as the color-changing reactive compound, a green-colored ABTS-bioreceptor solution turns from green to a pale color.

12. The method according to claim 2, wherein a process of immobilizing the bioreceptor to a surface of the the available working electrode is carried out with EDC/NHS, or different suitable crosslinking chemical compounds.

13. The method according to claim 2, wherein heparan sulfate is used as the at least one GAG.

14. The method according to claim 3, wherein heparan sulfate is used as the at least one GAG.

15. The method according to claim 2, wherein the at least one GAG is at least one of polysaccharides of heparin, chondroitin-4-sulfate, chondroitin-4,6-sulfate, and dermatan sulfate.

16. The method according to claim 3, wherein the at least one GAG is at least one of polysaccharides of heparin, chondroitin-4-sulfate, chondroitin-4,6-sulfate, and dermatan sulfate.

17. The method according to claim 7, wherein in a case of using gold nanoparticles as the color-changing reactive compound, a red-colored gold nanoparticle-bioreceptor solution turns into different shades of red, or purple.

18. The method according to claim 8, wherein in a case of using gold nanoparticles as the color-changing reactive compound, a red-colored gold nanoparticle-bioreceptor solution turns into different shades of red, or purple.

19. The method according to claim 7, wherein in a case of using a TMB+H₂O₂+oxidizing reagent mixture as the color-changing reactive compound, a blue-colored TMB-bioreceptor solution turns from blue into a pale color.

20. The method according to claim 8, wherein in a case of using a TMB+H₂O₂+oxidizing reagent mixture as the color-changing reactive compound, a blue-colored TMB-bioreceptor solution turns from blue into a pale color.
